(19) United States
(12) Patent Application Publication
(10) Pub. No.: US 2020/0188271 A1
(43) Pub. Date: Jun. 18, 2020

(54) SPRAYABLE COSMETIC COMPOSITION

(71) Applicant: Henkel AG & Co. KGaA, Duesseldorf (DE)

(72) Inventors: DIANE METTEN, Hamburg (DE); THORSTEN KNAPPE, Schenefeld (DE); TIM BETHGE, Oststeinbek (DE); RENE SCHEFFLER, Ellerau (DE); ROLF BAYERSDOERFER, Hamburg (DE)

(73) Assignee: Henkel AG & Co. KGaA, Duesseldorf (DE)

(21) Appl. No.: 16/689,626

(22) Filed: Nov. 20, 2019

(30) Foreign Application Priority Data
Dec. 18, 2018 (DE) 10 2018 222 104.9

(51) Int. Cl. A61K 8/73; A61K 8/81; A61K 8/9789; A61K 8/34; A61Q 5/06; A61K 8/04

(52) U.S. Cl. CPC A61K 8/731; A61K 8/8117; A61K 8/8129; A61K 2800/87; A61K 8/34; A61Q 5/06; A61K 8/046; A61K 8/9789

(57) ABSTRACT

A cosmetic composition for treating keratin fibres, contains:

(a) fibres

(b) ethanol

(c) at least one polymer, comprising an acrylate copolymer, and

(d) propellant.

### CROSS-REFERENCE TO RELATED APPLICATION

This application claims priority to German Patent Application No. 10 2018 222 104.9, filed Dec. 18, 2018, which is incorporated herein by reference in its entirety.

### TECHNICAL FIELD

The present application relates to sprayable cosmetic compositions for the treatment of keratin fibres, which compositions, in addition to a solvent and a propellant, also contain fibres. These compositions are suitable for a wide range of cosmetic uses, but in particular for the temporary reshaping of keratin fibres. The application also relates to the cosmetic use of these compositions and to hair cosmetic methods employing these compositions.

### BACKGROUND

Styling products for reshaping keratinous fibres have long been known and are used in various embodiments in order to structure, refresh and fix hairstyles, which in the case of many hair types can be achieved only with the use of firming active substances. Here, both hair treatment products which are used to permanently shape and also those used to temporarily shape the hair play an important role. Temporary shaping that should have good hold without detriment to the healthy appearance of the hair, for example its shine, can be attained for example by hairsprays, hair waxes, hair gels, setting lotions, etc.

Corresponding compositions for temporary shaping usually contain synthetic polymers as shaping component. Preparations that contain a polymer can be applied to the hair by employing propellant gases or by a pump mechanism. Hair gels and hair waxes, by contrast, generally are not applied directly to the hair, but instead are distributed through the hair by employing a comb or by the hands.

Users of styling products who have thin hair and/or little hair volume wish to achieve thicker hair or greater hair body, in particular when they run their fingers through their hair. At the same time, the hair should look and feel natural.

Hairsprays fix the keratin fibres in a desired form, but do not give the look or feel of thickness and/or body. Dry shampoos can add volume to the hair, but have no styling properties. In addition, dry shampoos often leave behind residues on the keratin fibres.

### BRIEF SUMMARY

The object of the present disclosure was to provide sprayable hair cosmetic compositions for the cosmetic treatment of keratin fibres, which compositions, in comparison to conventional preparations, impart hold as well as thickness and body to the keratin fibres treated with them.

In various embodiments, a cosmetic composition for the cosmetic treatment of keratin fibres, comprises (a) fibres; (b) ethanol; (c) at least one polymer, comprising an acrylate copolymer; and (d) propellant. For example, in an embodiment, the acrylate copolymer is selected from the group of compounds with the INCI name Ethylhexyl Acrylate/Methyl Methacrylate Copolymer

In embodiments, the cosmetic composition comprises further polymers selected from the group consisting of polyvinyl acetate, a copolymer with the INCI name Castor Oil/IPDI Copolymer, polyurethanes, cellulose ethers, polyethylene glycols, a copolymer with the INCI name Acrylates/Hydroxyesters Acrylates Copolymer, copolymers of methylvinyl ether and maleic acid anhydride, and a copolymer with the INCI name Crotonic Acid/Vinyl C8-12 Isoalkyl Esters/VA/Bis-Vinyldimethicone Crosspolymer.

There is also provided a cosmetic product, comprising the cosmetic composition contained in an aerosol-dispensing container and a method for increasing the thickness of keratin fibres, comprising applying the cosmetic composition to the keratin fibres.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It has been found that this object can be achieved by the addition of an acrylate copolymer to the sprayable cosmetic preparations.

The following is provided by employing the present disclosure:

1. A cosmetic composition for the cosmetic treatment of keratin fibres, said composition comprising:
(a) fibres
(b) ethanol
(c) at least one polymer, comprising an acrylate copolymer,
(d) propellant.
   2. The cosmetic composition according to point 1, wherein the cosmetic composition comprises an acrylate copolymer which is obtained by reacting ethylhexyl acrylate with methyl methacrylate.
   3. The cosmetic composition according to point 1 or 2, wherein the cosmetic composition comprises an acrylate copolymer, which is selected from the group of compounds with the INCI name Ethylhexyl Acrylate/Methyl Methacrylate Copolymer
   4. The cosmetic composition according to any one of the preceding points, wherein the weight proportion of the fibres in the total weight of the cosmetic composition is from about 0.05 to about 5, preferably from about 0.1 to about 2% by weight, particularly preferably from about 0.1 to about 1% by weight, and in particular from about 0.1 to about 0.5% by weight.
   5. The cosmetic composition according to any one of the preceding points, wherein the fibres are selected from the group of fibres produced from natural fibre materials.
   6. The cosmetic composition according to any one of the preceding points, wherein the fibres are selected from the group of plant fibres, in particular cellulose fibres, preferably cotton fibres, flax fibres, bamboo fibres and kapok fibres.
   7. The cosmetic composition according to any one of the preceding points, wherein the fibres are selected from the group of fibres produced from synthetic fibre materials.
   8. The cosmetic composition according to any one of the preceding points, wherein the fibres are selected from the group of polyamide fibres, polyester fibres, polylactic acid fibres and polyhydroxyalkanoate fibres.
   9. The cosmetic composition according to any one of the preceding points, wherein the fibres have a length of from about 1 to about 200 μm, preferably of from about 2 to about 150 μm, and in particular of from about 5 to about 100 μm.
   10. The cosmetic composition according to any one of the preceding points, wherein the weight proportion of the ethanol in the total weight of the cosmetic composition is from about 10 to about 40% by weight, preferably from about 15 to about 35% by weight, and in particular from about 20 to about 30% by weight.
   11. The cosmetic composition according to any one of the preceding points, wherein the weight proportion of the at least one polymer in the total weight of the cosmetic composition is from about 0.1 to about 10% by weight, preferably from about 0.5 to about 8% by weight, and in particular from about 1 to about 4% by weight.
   12. The cosmetic composition according to any one of the preceding points, wherein the at least one polymer contains a further polymer selected from the group including polyvinyl acetate, a copolymer with the INCI name Castor Oil/IPDI Copolymer, polyurethanes, cellulose ethers, siloxanes, polyethylene glycols, a copolymer with the INCI name Acrylates/Hydroxyesters Acrylates Copolymer, copolymers of methylvinyl ether and maleic acid anhydride, a copolymer with the INCI name Crotonic Acid/Vinyl C8-12 Isoalkyl EstersNA/Bis-Vinyldimethicone Crosspolymer and mixtures thereof.
   13. The cosmetic composition according to point 12, wherein the composition comprises a cellulose ether as further polymer.
   14. The cosmetic composition according to point 12, wherein the composition comprises a cellulose ether and polyvinyl acetate as further polymer.
   15. The cosmetic composition according to point 12, wherein the composition comprises a cellulose ether, polyvinyl acetate and a copolymer with the INCI name Castor Oil/IPDI Copolymer as further polymer.
   16. The cosmetic composition according to point 12, wherein the composition comprises a cellulose ether, polyvinyl acetate, a copolymer with the INCI name Castor Oil/IPDI Copolymer and polyethylene glycol as further polymer.
   17. The cosmetic composition according to point 12, wherein the composition comprises a cellulose ether and a copolymer with the INCI name Castor Oil/IPDI Copolymer as further polymer.
   18. The cosmetic composition according to point 12, wherein the composition comprises a cellulose ether, polyvinyl acetate and a copolymer with the INCI name Crotonic Acid/Vinyl C8-12 Isoalkyl Esters/VA/Bis-Vinyldimethicone Crosspolymer as further polymer.
   19. The cosmetic composition according to point 12, wherein the composition comprises a cellulose ether, polyvinyl acetate and polyethylene glycol as further polymer.
   20. The cosmetic composition according to any one of the preceding points, wherein the cosmetic composition also contains an oligomer of a urethane diol.
   21. The cosmetic composition according to any one of the preceding points, wherein the cosmetic composition also contains a hydrophobised metal oxide powder.
   22. The cosmetic composition according to any one of the preceding points, wherein the weight proportion of the propellant in the total weight of the cosmetic composition is from about 40 to about 80% by weight, preferably from about 45 to about 75% by weight, and in particular from about 50 to about 70% by weight.
   23. The cosmetic composition according to any one of the preceding points, wherein the composition comprises a propellant from the group propane, propane/butane mixtures and dimethyl ether, in particular dimethyl ether.
   24. The cosmetic composition according to any one of the preceding points, wherein the composition comprises a propellant from the group of compressed gases, preferably selected from the group including air, nitrogen oxide, nitrogen, carbon dioxide, and mixtures thereof.
   25. A cosmetic product, comprising
a) a cosmetic composition according to any one of points 1 to 24
b) an aerosol-dispensing container.
   26. Use of a cosmetic composition according to any one of points 1 to 24 for temporarily reshaping keratin fibres.
   27. Use of a cosmetic composition according to any one of points 1 to 24 for increasing the thickness of keratin fibres treated with the composition.
   28. A method for temporarily reshaping keratin fibres, in particular human hair, in which the cosmetic composition according to any one of points 1 to 24 is applied to keratin fibres.
   29. A method for increasing the thickness of keratin fibres, in particular human hair, in which the cosmetic composition according to any one of points 15 to 17 is applied to keratin fibres.

The cosmetic composition contains fibres as its first essential constituent. The addition of these fibres influences the application properties of the sprayable hair cosmetic in an unanticipated manner. The spray pattern and the application of the sprayed cosmetic are more uniform. In a standardised test assembly the addition of the fibres at the same pressure in the spraying device and at the same distance from the surface that is to be sprayed additionally results in a surprising enlargement of the spray area that is achieved.

Oriented, flexible structures formed from fibre material with a ratio of length to diameter above about 3:1, preferably above about 5:1, and in particular above about 10:1 are suitable as fibres. Fibres cannot take up compressive forces in the longitudinal direction, but instead only tensile forces.

The length of fibres that are used with preference ranges from about 1 to about 200 μm, preferably from about 2 to about 150 μm, and in particular from about 5.0 to about 100 μm. Of course, however, fibres of greater fibre length, for example fibres with a length above about 200 μm or above about 300 μm, can also be used.

The fibre material can be of natural or synthetic origin. All fibres that are obtained from plant, animal or mineral material without any chemical modification are considered to be natural fibres.

Plant fibres occur in plants as a vascular bundle in the stalk, in the stem, in the bark or in the form of seed extensions. Plant fibres generally consist predominantly of cellulose. Corresponding fibres are amalgamated under the name cellulose fibres.

In accordance with common subdivisioning, a distinction is made in the case of plant fibres between seed fibres, bast fibres, leaf fibres and fruit fibres. The group of seed fibres for example includes cotton fibres, kapok, crown flower seed fibres, or populus fruit seed fibres. The group of bast fibres includes, amongst other things, bamboo fibres, stinging nettles, hemp fibres, jute, and flax fibres. Sisal is considered to belong to the leaf fibres, whereas coconut fibres belong to the group of fruit fibres. Bamboo fibres are also suitable.

In the case of animal fibres a distinction can be made, amongst other things, between fibres from silk glands and fibres originating from hair follicles. Fibres from silk glands include silk, for example. The group of fibres originating from hair follicles includes, amongst other things, wool, alpaca, camel hair, angora, cashmere, mohair, yak hair, goat hair, cattle hair or horsehair.

In a preferred embodiment the fibres used in the cosmetic compositions are selected from the group of fibres formed from natural fibre materials. The use of cellulose fibres, in particular cotton fibres, flax fibres and kapok fibres, is particularly preferred. The use of animal fibres from the group of silk fibres, cashmere fibres and wool fibres is also preferred. The use of cellulose fibres is particularly preferred.

Alternatively or in combination with the above-described natural fibres, fibres based on synthetic fibre materials can also be used in the cosmetic compositions.

The use of synthetic fibre materials obtained by modification of plant or animal starting materials is particularly preferred. The group of these preferred fibres includes, for example, fibres formed from viscose, polylactic acid, alginate, chitin or chitosan.

Further preferred synthetic fibre materials are polyamides, polyesters and polyhydroxyalkanoates.

Weight proportions of the fibres in the total weight of the cosmetic composition from about 0.05 to about 5, preferably from about 0.1 to about 2% by weight, particularly preferably from about 0.1 to about 1% by weight, and in particular from about 0.1 to about 0.5% by weight have proven to be particularly advantageous for the technical and cosmetic effect.

As second essential constituent, the cosmetic compositions contain ethanol. The weight proportion of the ethanol in the total weight of the cosmetic composition is preferably from about 10 to about 50% by weight, preferably from about 15 to about 45% by weight, and in particular from about 20 to about 40% by weight. The fibres are suspended in the ethanol.

The third essential constituent of the cosmetic composition is the at least one polymer. For the applicability and cosmetic effect of the compositions, it has proven to be advantageous to limit the weight proportion of the polymer in the total weight of the composition to from about 0.1 to about 10% by weight, preferably from about 0.5 to about 8% by weight, and in particular from about 1 to about 4% by weight.

Polymers are used in the cosmetic compositions for example on account of their firming and/or film-forming properties. The use of film-forming polymers is particularly preferred on account of their cosmetic effect. The use of polymers with a thickening effect, with a hair-texturising effect, with a hair-nourishing effect and/or with a hair-conditioning effect may also be advantageous.

The at least one polymer mandatorily comprises an acrylate copolymer. An acrylate copolymer within the scope of this application comprises at least two different monomers, one of which is mandatorily an acrylate or methacrylate. Acrylate is a collective term for substances which are exemplified chemically by the acryl group (CH₂=CH—COR) (depending on R, it may be acrylic acid or the salts or esters of acrylic acid). Methacrylates have a methacrylic group accordingly (CH₃CH=CH—COR).

It is particularly preferred if the at least one polymer contains an acrylate copolymer which is obtained by reacting ethylhexyl acrylate with methyl methacrylate. Preferred acrylate copolymers are selected from the group with the INCI name Ethylhexyl Acrylate/Methyl Methacrylate Copolymer.

Such an acrylate copolymer is obtainable from EOC, for example under the name “Eurocryl BC 4365 HE” (INCI: Ethylhexyl Acrylate/Methyl Methacrylate Copolymer, active substance: about 50% by weight in water).

The at least one polymer may comprise further polymers. Permanently and also temporarily cationic, anionic, non-ionic or amphoteric polymers are suitable as further polymers. The further polymers may be of synthetic or natural origin.

Examples of further polymers are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVPNA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer.

The further polymer is particularly preferably selected from the group of non-ionic or temporarily cationic polymers. Suitable polymers are, for example:
Polyvinyl acetates, as are sold for example under the name Vinnapas® (Wacker Chemie).
Cellulose ethers, such as hydroxypropyl cellulose (HPC), hydroxyethyl cellulose (HEC) and hydroxypropyl methylcellulose (HPMC), as sold for example under the trade name Affinisol by Dow Pharma Solutions.
Polyurethanes, as are sold for example under the name Polyderm PPI-CO (INCI: Castor Oil/IPDI Copolymer) by Alzo International.
Copolymers of methyl vinyl ether and maleic acid anhydride, in particular esters thereof, as are sold for example under the name Omnirez 2000 (INCI: Ethyl Ester of PVM/MA Copolymer) by Ashland.
Further acrylate copolymers, such as in particular a copolymer with the INCI name Acrylates/Hydroxyesters Acrylates Copolymer.
Silicone organic polymer compound, such as a compound with the INCI name Crotonic Acid/Vinyl C8-12 Isoalkyl Esters/VA/Bis-Vinyldimethicone Crosspolymer, which is provided under the name BELSIL® P 1101 as about 50% solution in denatured ethanol by Wacker.
Polyethylene glycols, such as in particular PEG-20, PEG-32, PEG-33, PEG-40, PEG-45, PEG-55, PEG-60, PEG-75, PEG-80, PEG-90, PEG-100, PEG-135, PEG-150, PEG-180, PEG-200, PEG-220, PEG-240, PEG-350, PEG-400, PEG-500, PEG-800, PEG-2M, PEG-5M, PEG-7M, PEG-9M, PEG-14M, PEG-20M, PEG-23M, PEG-25M, PEG-45M, PEG-65M, PEG-90M, PEG-115M, PEG-160M or PEG-180M.
   Cosmetic compositions which, besides the acrylate copolymer, also comprise a further polymer selected from the group consisting of polyvinyl acetate, a copolymer with the INCI name Castor Oil/IPDI Copolymer, polyurethanes, cellulose ethers, siloxanes, polyethylene glycols, a copolymer with the INCI name Acrylates/Hydroxyesters Acrylates Copolymer, copolymers of methylvinyl ether and maleic acid anhydride, a copolymer with the INCI name Crotonic Acid/Vinyl C8-12 Isoalkyl Esters/VA/Bis-Vinyldimethicone Crosspolymer and mixtures thereof are preferred.

It is particularly preferred that the composition comprises a cellulose ether, preferably hydroxypropyl methylcellulose (HPMC), as further polymer.

Accordingly, preferred cosmetic compositions are those which have one of the following combinations of further polymers:
a cellulose ether and polyvinyl acetate,
a cellulose ether, polyvinyl acetate and a copolymer with the INCI name Castor Oil/IPDI Copolymer,
a cellulose ether, polyvinyl acetate, a copolymer with the INCI name Castor Oil/IPDI Copolymer, and polyethylene glycol,
a cellulose ether and a copolymer with the INCI name Castor Oil/IPDI Copolymer,
a cellulose ether, polyvinyl acetate and a copolymer with the INCI name Crotonic Acid/Vinyl C8-12 Isoalkyl Esters/VA/Bis-Vinyldimethicone Crosspolymer, or
a cellulose ether, polyvinyl acetate and polyethylene glycol.

The physical properties of the composition, but also the cosmetic properties on/at the hair can be set by the combination of a very wide range of polymers and their amounts. For example, compositions which impart volume and body on the hair treated with them, or compositions which additionally also have a texturising effect and generate more hold can be obtained.

A copolymer with the INCI name Crotonic Acid/Vinyl C8-12 Isoalkyl Esters/VA/Bis-Vinyldimethicone Crosspolymer for example combines the strong hold properties of a hair-styling resin with typical silicone properties, such as softness and silkiness. Compositions containing this polymer have a texturising effect. The amount of copolymer with the INCI name Crotonic Acid/Vinyl C8-12 Isoalkyl Esters/VA/Bis-Vinyldimethicone Crosspolymer may be from about 0.1 to about 2% by weight, in relation to the total amount of composition.

It is accordingly preferred if the cosmetic composition comprises a copolymer with the INCI name Crotonic Acid/Vinyl C8-12 Isoalkyl Esters/VA/Bis-Vinyldimethicone Crosspolymer as further polymer.

The use of polyethylene glycols, such as PEG-32, also leads to a texturising effect of the compositions containing them. The amount of polyethylene glycol, in particular PEG-32, may be from about 0.1 to about 2% by weight, in relation to the total amount of composition.

Accordingly, it is preferred if the composition comprises a polyethylene glycol, preferably with a molar mass greater than about 880 g/mol, as further polymer.

The use of polyurethanes, such as a copolymer with the INCI name Castor Oil/IPDI Copolymer, leads to a noticeable increase in hair body. The amount of polyurethane may be from about 0.1 to about 9.9% by weight, in relation to the total amount of composition.

It is particularly preferred if the composition comprises a copolymer with the INCI name Castor Oil/IPDI Copolymer as further polymer.

The amount of cellulose ether, in particular of hydroxypropyl methylcellulose, may be from about 0.05 to about 2% by weight, in relation to the total amount of composition.

The amount of polyvinyl acetate (homopolymer of vinyl acetate) may be from about 0.1 to about 9.9% by weight, in relation to the total amount of composition.

The amount of copolymer of methyl vinyl ether and maleic acid anhydride (INCI: PVM/MA Copolymer), in particular the ethyl ester of copolymers (INCI: Ethyl Ester of PVM/MA Copolymer) may be from about 0.1 to about 9.9% by weight, in relation to the total amount of composition.

A fourth essential constituent of the cosmetic compositions is the propellant. It has proven to be particularly advantageous from a cosmetic and technical viewpoint of the weight proportion of the propellant in the total weight of the composition is from about 40 to about 80% by weight, preferably from about 45 to about 75% by weight, and in particular from about 50 to about 75% by weight.

Preferred propellants (propellant gases) are selected from propane, propene, n-butane, iso-butane, iso-butene, n-pentane, pentene, iso-pentane, iso-pentene, methane, ethane, dimethyl ether, nitrogen, air, oxygen, nitrous oxide, dichlorofluoromethane, chlorodifluoromethane, chlorofluoromethane, 1,1,2,2-tetrachloro-1-fluoroethane, 1,1,1,2-tetrachloro -2-fluoroethane, 1,2,2-trichloro-1,1-difluoroethane, 1,1,2-trichloro-1,2-difluoroethane, 1,1,1-trichloro-2,2-difluoroethane, 2,2-dichloro-1,1,1-trifluoroethane, 1,2-dichloro -1,1,2-trifluoroethane, 2-chloro-1,1,1,2-tetrafluoroethane, 1-chloro-1,1,2,2-tetrafluoroethane, 1,1,2-trichloro-2-fluoroethane, 1,2-dichloro-1,2-difluoroethane, 1,2-dichloro -1,1-difluoroethane, 1-chloro-1,2,2-trifluoroethane, 2-chloro-1,1,1-trifluoroethane, 1-chloro -1,1,2-trifluoroethane, 1,2-dichloro-1-fluoroethane, 1,1-dichloro-1-fluoroethane, 2-chloro -1,1-difluoroethane, 1-chloro-1,1-difluoroethane, 1-chloro-2-fluoroethane, 1-chloro-1-fluoroethane, 2-chloro-1,1-difluoroethene, 1,1,1,3-tetrafluoroethane, heptafluoro-n-propane, perfluoroethane, monochlorodifluoromethane, 1,1-difluoroethane, more specifically either individually or in combination.

The use of propane, propane/butane mixtures or dimethyl ether is preferred, and the use of dimethyl ether is particularly preferred.

The composition of some preferred cosmetic compositions can be deduced from the following overview 1 (specified amounts in % by weight are in relation to the total weight of the cosmetic composition).

| | Formula 1 | Formula 2 | Formula 3 | Formula 4 | Formula 5 |
|---|---|---|---|---|---|
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising an | 0.1 to 10 | 0.1 to 10 | 0.5 to 8 | 1 to 4 | 1 to 4 |
| acrylate copolymer | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 1a | Formula 2a | Formula 3a | Formula 4a | Formula 5a |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 1b | Formula 2b | Formula 3b | Formula 4b | Formula 5b |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer and | | | | | |
| Hydroxypropyl | | | | | |
| methylcellulose | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 1c | Formula 2c | Formula 3c | Formula 4c | Formula 5c |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer, | | | | | |
| Hydroxypropyl | | | | | |
| methylcellulose and | | | | | |
| Castor Oil/IPDI | | | | | |
| Copolymer | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 6 | Formula 7 | Formula 8 | Formula 9 | Formula 10 |
| Cellulose fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising an | 0.1 to 10 | 0.1 to 10 | 0.5 to 8 | 1 to 4 | 1 to 4 |
| acrylate copolymer | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 6a | Formula 7a | Formula 8a | Formula 9a | Formula 10a |
| Cellulose fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 6b | Formula 7b | Formula 8b | Formula 9b | Formula 10b |
| Cellulose fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer and | | | | | |
| Hydroxypropyl | | | | | |
| methylcellulose | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 6c | Formula 7c | Formula 8c | Formula 9c | Formula 10c |
| Cellulose fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer, | | | | | |
| Hydroxypropyl | | | | | |
| methylcellulose and | | | | | |
| Castor Oil/IPDI | | | | | |
| Copolymer | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 11 | Formula 12 | Formula 13 | Formula 14 | Formula 15 |
| Fibres with a fibre length | 0.05 to 5 | 0.1 to 2 | 0.1 to 2 | 0.1 to 1 | 0.1 to 0.5 |
| between 1.0 and 200 μm | | | | | |
| Ethanol | 10 to 40 | 10 to 40 | 15 to 35 | 20 to 30 | 20 to 30 |
| Polymer, comprising an | 0.1 to 10 | 0.1 to 10 | 0.5 to 8 | 1 to 4 | 1 to 4 |
| acrylate copolymer | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 11a | Formula 12a | Formula 13a | Formula 14a | Formula 15a |
| Fibres with a fibre length | 0.05 to 5 | 0.1 to 2 | 0.1 to 2 | 0.1 to 1 | 0.1 to 0.5 |
| between 1.0 and 200 μm | | | | | |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 11b | Formula 12b | Formula 13b | Formula 14b | Formula 15b |
| Fibres with a fibre length | 0.05 to 5 | 0.1 to 2 | 0.1 to 2 | 0.1 to 1 | 0.1 to 0.5 |
| between 1.0 and 200 μm | | | | | |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer and | | | | | |
| Hydroxypropyl | | | | | |
| methylcellulose | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 11c | Formula 12c | Formula 13c | Formula 14c | Formula 15c |
| Fibres with a fibre length | 0.05 to 5 | 0.1 to 2 | 0.1 to 2 | 0.1 to 1 | 0.1 to 0.5 |
| between 1.0 and 200 μm | | | | | |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer, | | | | | |
| Hydroxypropyl | | | | | |
| methylcellulose and | | | | | |
| Castor Oil/IPDI | | | | | |
| Copolymer | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 16 | Formula 17 | Formula 18 | Formula 19 | Formula 20 |
| Cellulose fibres with a | 0.05 to 5 | 0.1 to 2 | 0.1 to 2 | 0.1 to 1 | 0.1 to 0.5 |
| fibre length between 1.0 | | | | | |
| and 200 μm | | | | | |
| Ethanol | 10 to 40 | 10 to 40 | 15 to 35 | 20 to 30 | 20 to 30 |
| Polymer, comprising an | 0.1 to 10 | 0.1 to 10 | 0.5 to 8 | 1 to 4 | 1 to 4 |
| acrylate copolymer | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 16a | Formula 17a | Formula 18a | Formula 19 | Formula 20a |
| Cellulose fibres with a | 0.05 to 5 | 0.1 to 2 | 0.1 to 2 | 0.1 to 1 | 0.1 to 0.5 |
| fibre length between 1.0 | | | | | |
| and 200 μm | | | | | |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 16b | Formula 17b | Formula 18b | Formula 19b | Formula 20b |
| Cellulose fibres with a | 0.05 to 5 | 0.1 to 2 | 0.1 to 2 | 0.1 to 1 | 0.1 to 0.5 |
| fibre length between 1.0 | | | | | |
| and 200 μm | | | | | |
| Ethanol | 10 to 40 | 10 to 40 | 15 to 35 | 20 to 30 | 20 to 30 |
| Polymer. comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer and | | | | | |
| Hydroxypropyl | | | | | |
| methylcellulose | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 16c | Formula 17c | Formula 18c | Formula 19c | Formula 20c |
| Cellulose fibres with a | 0.05 to 5 | 0.1 to 2 | 0.1 to 2 | 0.1 to 1 | 0.1 to 0.5 |
| fibre length between 1.0 | | | | | |
| and 200 μm | | | | | |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer, | | | | | |
| Hydroxypropyl | | | | | |
| methylcellulose and | | | | | |
| Castor Oil/IPDI | | | | | |
| Copolymer | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 21 | Formula 22 | Formula 23 | Formula 24 | Formula 25 |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising an | 0.1 to 10 | 0.1 to 10 | 0.5 to 8 | 1 to 4 | 1 to 4 |
| acrylate copolymer | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 21a | Formula 22a | Formula 23a | Formula 24a | Formula 25a |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 21b | Formula 22b | Formula 23b | Formula 24b | Formula 25b |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer and | | | | | |
| Hydroxypropyl | | | | | |
| methylcellulose | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 21c | Formula 22c | Formula 23c | Formula 24c | Formula 25c |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer, | | | | | |
| Hydroxypropyl | | | | | |
| methylcellulose and | | | | | |
| Castor Oil/IPDI | | | | | |
| Copolymer | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 26 | Formula 27 | Formula 28 | Formula 29 | Formula 30 |
| Cellulose fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising an | 0.1 to 10 | 0.1 to 10 | 0.5 to 8 | 1 to 4 | 1 to 4 |
| acrylate copolymer | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 26a | Formula 27a | Formula 28a | Formula 29a | Formula 30a |
| Cellulose fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 26b | Formula 27b | Formula 28b | Formula 29b | Formula 30b |
| Cellulose fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer and | | | | | |
| Hydroxypropyl | | | | | |
| methylcellulose | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 26c | Formula 27c | Formula 28c | Formula 29c | Formula 30c |
| Cellulose fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer, | | | | | |
| Hydroxypropyl | | | | | |
| methylcellulose and | | | | | |
| Castor Oil/IPDI | | | | | |
| Copolymer | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 31 | Formula 32 | Formula 33 | Formula 34 | Formula 35 |
| Fibres with a fibre length | 0.05 to 5 | 0.1 to 2 | 0.1 to 2 | 0.1 to 1 | 0.1 to 0.5 |
| between 1.0 and 200 μm | | | | | |
| Ethanol | 10 to 40 | 15 to 35 | 15 to 35 | 20 to 30 | 20 to 30 |
| Polymer, comprising an | 0.1 to 10 | 0.1 to 10 | 0.5 to 8 | 1 to 4 | 1 to 4 |
| acrylate copolymer | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 31a | Formula 32a | Formula 33a | Formula 34a | Formula 35a |
| Fibres with a fibre length | 0.05 to 5 | 0.1 to 2 | 0.1 to 2 | 0.1 to 1 | 0.1 to 0.5 |
| between 1.0 and 200 μm | | | | | |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 31b | Formula 32b | Formula 33b | Formula 34b | Formula 35b |
| Fibres with a fibre length | 0.05 to 5 | 0.1 to 2 | 0.1 to 2 | 0.1 to 1 | 0.1 to 0.5 |
| between 1.0 and 200 μm | | | | | |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer and | | | | | |
| Hydroxypropyl | | | | | |
| methylcellulose | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 31c | Formula 32c | Formula 33c | Formula 34c | Formula 35c |
| Fibres with a fibre length | 0.05 to 5 | 0.1 to 2 | 0.1 to 2 | 0.1 to 1 | 0.1 to 0.5 |
| between 1.0 and 200 μm | | | | | |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer, | | | | | |
| Hydroxypropyl | | | | | |
| methylcellulose and | | | | | |
| Castor Oil/IPDI | | | | | |
| Copolymer | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 36 | Formula 37 | Formula 38 | Formula 39 | Formula 40 |
| Cellulose fibres with a | 0.05 to 5 | 0.1 to 2 | 0.1 to 2 | 0.1 to 1 | 0.1 to 0.5 |
| fibre length between 1.0 | | | | | |
| and 200 μm | | | | | |
| Ethanol | 10 to 40 | 10 to 40 | 15 to 35 | 20 to 30 | 20 to 30 |
| Polymer, comprising an | 0.1 to 10 | 0.1 to 10 | 0.5 to 8 | 1 to 4 | 1 to 4 |
| acrylate copolymer | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 36a | Formula 37a | Formula 38a | Formula 39a | Formula 40a |
| Cellulose fibres with a | 0.05 to 5 | 0.1 to 2 | 0.1 to 2 | 0.1 to 1 | 0.1 to 0.5 |
| fibre length between 1.0 | | | | | |
| and 200 μm | | | | | |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 36b | Formula 37b | Formula 38b | Formula 39b | Formula 40b |
| Cellulose fibres with a | 0.05 to 5 | 0.1 to 2 | 0.1 to 2 | 0.1 to 1 | 0.1 to 0.5 |
| fibre length between 1.0 | | | | | |
| and 200 μm | | | | | |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer and | | | | | |
| Hydroxypropyl | | | | | |
| methylcellulose | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |
| | Formula 36c | Formula 37c | Formula 38c | Formula 39c | Formula 40c |
| Cellulose fibres with a | 0.05 to 5 | 0.1 to 2 | 0.1 to 2 | 0.1 to 1 | 0.1 to 0.5 |
| fibre length between 1.0 | | | | | |
| and 200 μm | | | | | |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer, | | | | | |
| Hydroxypropyl | | | | | |
| methylcellulose and | | | | | |
| Castor Oil/IPDI | | | | | |
| Copolymer | | | | | |
| Dimethyl ether | 40 to 80 | 45 to 75 | 45 to 75 | 50 to 70 | 50 to 70 |
| Optional additives | to 100 | to 100 | to 100 | to 100 | to 100 |

Particularly preferred cosmetic compositions also contain a hydrophobised metal oxide. As contemplated herein “hydrophobised” is understood to mean metal oxides which have been modified at least at the surface of the particles in such a way that the modified particle is wetted to a lesser extent by water than the non-modified particle. In particular, silanised, hydrophobised metal oxides are preferred. At least one representative of the group formed from silanes, halosilanes, alkoxysilanes, and silazanes is preferably suitable as reagent for silanising the metal oxide. Preferably suitable hydrophobised metal oxides are selected from at least one representative of the group formed from hydrophobised silicates, hydrophobised aluminium silicates, hydrophobised titanium dioxide, and hydrophobised silicon dioxide. Hydrophobised silicates have proven to be particularly suitable, wherein fumed silicas post-treated with polydimethyl siloxane have particular advantages. Preferred compositions contain, as hydrophobised metal oxide powder, a hydrophobised silicon dioxide, preferably a fumed silica acid post-treated with dimethyl dichlorosilane. Corresponding metal oxides with the INCI name “Silica Dimethyl Silylate” are sold for example by the company Evonik under the trade name Aersoil® R972.

Preferred compositions contain the hydrophobised metal oxide powder—in relation to its total weight—in amounts of from about 0.1 to about 2% by weight.

The composition of further preferred cosmetic compositions can be deduced from the following overview 2 (specified amounts in % by weight are in relation to the total weight of the cosmetic composition).

| | Formula 41 | Formula 42 | Formula 43 | Formula 44 | Formula 45 |
|---|---|---|---|---|---|
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising an | 0.1 to 10 | 0.1 to 10 | 0.5 to 8 | 1 to 4 | 1 to 4 |
| acrylate copolymer | | | | | |
| hydrophobised metal | 0.1 to 2 | 0.1 to 2 | 0.1 to 2 | 0.1 to 2 | 0.1 to 2 |
| oxide | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 41a | Formula 42a | Formula 43a | Formula 44a | Formula 45a |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer | | | | | |
| hydrophobised metal | 0.1 to 2 | 0.1 to 2 | 0.1 to 2 | 0.1 to 2 | 0.1 to 2 |
| oxide | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 41b | Formula 42b | Formula 43b | Formula 44b | Formula 45b |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer and | | | | | |
| Hydroxypropyl | | | | | |
| methylcellulose | | | | | |
| hydrophobised metal oxide | 0.1 to 2 | 0.1 to 2 | 0.1 to 2 | 0.1 to 2 | 0.1 to 2 |
| Propellant | 40 to 80 | 45 to 75 | 45 to 75 | 50 to 70 | 50 to 70 |
| Optional additives | to 100 | to 100 | to 100 | to 100 | to 100 |
| | Formula 41c | Formula 42c | Formula 43c | Formula 44c | Formula 45c |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer, | | | | | |
| Hydroxypropyl | | | | | |
| methylcellulose and | | | | | |
| Castor Oil/IPDI | | | | | |
| Copolymer | | | | | |
| hydrophobised metal | 0.1 to 2 | 0.1 to 2 | 0.1 to 2 | 0.1 to 2 | 0.1 to 2 |
| oxide | | | | | |
| Propellant | 40 to 80 | 45 to 75 | 45 to 75 | 50 to 70 | 50 to 70 |
| Optional additives | to 100 | to 100 | to 100 | to 100 | to 100 |
| | Formula 46 | Formula 47 | Formula 48 | Formula 49 | Formula 50 |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising an | 0.1 to 10 | 0.1 to 10 | 0.5 to 8 | 1 to 4 | 1 to 4 |
| acrylate copolymer | | | | | |
| Silica Dimethyl Silylate | 0.1 to 2 | 0.1 to 2 | 0.1 to 2 | 0.1 to 2 | 0.1 to 2 |
| Propellant | 40 to 80 | 45 to 75 | 45 to 75 | 50 to 70 | 50 to 70 |
| Optional additives | to 100 | to 100 | to 100 | to 100 | to 100 |
| | Formula 46a | Formula 47a | Formula 48a | Formula 49a | Formula 50a |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer | | | | | |
| Silica Dimethyl Silylate | 0.1 to 2 | 0.1 to 2 | 0.1 to 2 | 0.1 to 2 | 0.1 to 2 |
| Propellant | 40 to 80 | 45 to 75 | 45 to 75 | 50 to 70 | 50 to 70 |
| Optional additives | to 100 | to 100 | to 100 | to 100 | to 100 |
| | Formula 46b | Formula 47b | Formula 48b | Formula 49b | Formula 50b |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer and | | | | | |
| Hydroxypropyl | | | | | |
| methylcellulose | | | | | |
| Silica Dimethyl Silylate | 0.1 to 2 | 0.1 to 2 | 0.1 to 2 | 0.1 to 2 | 0.1 to 2 |
| Propellant | 40 to 80 | 45 to 75 | 45 to 75 | 50 to 70 | 50 to 70 |
| Optional additives | to 100 | to 100 | to 100 | to 100 | to 100 |
| | Formula 46c | Formula 47c | Formula 48c | Formula 49c | Formula 50c |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer, | | | | | |
| Hydroxypropyl | | | | | |
| methylcellulose and | | | | | |
| Castor Oil/IPDI | | | | | |
| Copolymer | | | | | |
| Silica Dimethyl Silylate | 0.1 to 2 | 0.1 to 2 | 0.1 to 2 | 0.1 to 2 | 0.1 to 2 |
| Propellant | 40 to 80 | 45 to 75 | 45 to 75 | 50 to 70 | 50 to 70 |
| Optional additives | to 100 | to 100 | to 100 | to 100 | to 100 |

Likewise, particularly preferred cosmetic compositions also contain an oligomer of a urethane diol.

K-Flex® UD-320-100 from King Industries is for example an aliphatic, low-molecular urethane diol oligomer which is obtainable with about 100% active substance content. Preferred compositions contain the oligomer of a urethane diol in amounts of from about 0.1 to about 5% by weight, and more preferably from about 1 to about 2% by weight, in relation to their total weight.

The composition of further preferred cosmetic compositions can be deduced from the following overview 3 (specified amounts in % by weight are in relation to the total weight of the cosmetic composition).

| | Formula 51 | Formula 52 | Formula 53 | Formula 54 | Formula 55 |
|---|---|---|---|---|---|
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising an | 0.1 to 10 | 0.1 to 10 | 0.5 to 8 | 1 to 4 | 1 to 4 |
| acrylate copolymer | | | | | |
| Oligomer of a urethane diol | 0.1 to 5 | 0.1 to 5 | 0.1 to 5 | 1 to 2 | 1 to 2 |
| Propellant | 40 to 80 | 45 to 75 | 45 to 75 | 50 to 70 | 50 to 70 |
| Optional additives | to 100 | to 100 | to 100 | to 100 | to 100 |
| | Formula 51a | Formula 52a | Formula 53a | Formula 54a | Formula 55a |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer | | | | | |
| Oligomer of a urethane diol | 0.1 to 5 | 0.1 to 5 | 0.1 to 5 | 1 to 2 | 1 to 2 |
| Propellant | 40 to 80 | 45 to 75 | 45 to 75 | 50 to 70 | 50 to 70 |
| Optional additives | to 100 | to 100 | to 100 | to 100 | to 100 |
| | Formula 51b | Formula 52b | Formula 53b | Formula 54b | Formula 55b |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer and | | | | | |
| Hydroxypropyl | | | | | |
| methylcellulose | | | | | |
| Oligomer of a urethane diol | 0.1 to 5 | 0.1 to 5 | 0.1 to 5 | 1 to 2 | Ito 2 |
| Propellant | 40 to 80 | 45 to 75 | 45 to 75 | 50 to 70 | 50 to 70 |
| Optional additives | to 100 | to 100 | to 100 | to 100 | to 100 |
| | Formula 51c | Formula 52c | Formula 53c | Formula 54c | Formula 55c |
| Fibres | 0.05 to 5 | 0.1 to 2 | 0.1 to 2 | 0.1 to 1 | 0.1 to 0.5 |
| Ethanol | 10 to 40 | 10 to 40 | 15 to 35 | 20 to 30 | 20 to 30 |
| Polymer, comprising | 0.1 to 10 | 0.1 to 10 | 0.5 to 8 | 1 to 4 | 1 to 4 |
| Ethylhexyl Acrylate/ | | | | | |
| Methyl Methacrylate | | | | | |
| Copolymer, | | | | | |
| Hydroxypropyl | | | | | |
| methylcellulose and Castor | | | | | |
| Oil/IPDI Copolymer | | | | | |
| Oligomer of a urethane diol | 0.1 to 5 | 0.1 to 5 | 0.1 to 5 | 1 to 2 | 1 to 2 |
| Propellant | 40 to 80 | 45 to 75 | 45 to 75 | 50 to 70 | 50 to 70 |
| Optional additives | to 100 | to 100 | to 100 | to 100 | to 100 |

Preferred cosmetic compositions also contain

(e) from 0 to about 10% by weight dye(s)
(f) from 0 to about 10% by pigment(s)
on the proviso that the sum of the amounts of ingredients (e) and (f) is from about 0.0001 to about 10% by weight, in relation to the total weight of the cosmetic composition.

In other words, the compositions can contain 0% by weight of dyes (in that case at least one pigment must be present in an amount of at least about 0.0001% by weight), the dye content however can also be about 10% by weight (in that case, pigment can no longer be present). Regardless of whether
only one dye,
a plurality of dyes,
only one pigment,
a plurality of pigments,
one dye and a plurality of pigments
a plurality of dyes and one pigment
a plurality of dyes and a plurality of pigments is/are used, the total amount of all dyes and pigments (also referred to as total amount of colour-giving components) in the cosmetic composition is from about 0.0001 to about 10% by weight, in relation to the total weight of the cosmetic composition.

It has been found that such coloured cosmetic compositions and/or those that have been mixed with dyes or pigments not only increase hair volume and increase body and give a feel of “having more hair”, but the colouring of the products can at the same time cover bald spots without the colour rubbing off on the hands or clothing or pillows.

Preferred cosmetic compositions contain from about 0.0001 to about 10% by weight, preferably from about 0.01 to about 9.5% by weight, more preferably from about 0.05 to about 9% by weight, even more preferably from about 0.1 to about 8.5% by weight, and in particular from about 0.25 to about 8% by weight of dye(s), in each case in relation to the total weight of the cosmetic composition.

Dyes are colourants, i.e. colour-giving substances, which in contrast to pigments are soluble in the application medium (for example in water, oils or other solvents).

A dye or a mixture of dyes can be selected depending on the desired colour, colour intensity and fastness properties of the resultant colouring. All dyes that are conventional and commercially obtainable in the field of cosmetics can be used without difficulty within the scope of the present disclosure.

Cosmetic compositions that are likewise preferred contain from about 0.0001 to about 10% by weight, preferably from about 0.01 to about 9.5% by weight, more preferably from about 0.05 to about 9% by weight, even more preferably from about 0.1 to about 8.5% by weight, and in particular from about 0.25 to about 8% by weight of pigment(s), in each case in relation to the total weight of the cosmetic composition.

All types of water-insoluble pigments, for example natural inorganic pigments (also referred to as mineral pigments) are suitable in principle. These pigments contain predominantly sulfides and oxides. Examples of such pigments are ochre (Fe(OOH); Pigment Yellow 43), burnt sienna (Fe₂O₃; Pigment Red 102), umbra (Fe₂O₃×MnO₂; Pigment Brown 7:×), cinnabar (β-HgS, PR 106), lapis lazuli (ultramarine, Na₆Al₆Si₆O₂₄×Na₂Sn; Pigment Blue 29), azurite (basic copper carbonate, Cu₃[OH/CO₃]₂; PB 30), green earth (FeO-containing silicate; Pigment Green 23), malachite (Cu₂[(OH)₂, CO₃]) and carbon black (carbon (graphite), Pigment Black 9). With regard to the avoidance of undesirable visible residues or grey films or the water-resistant temporary colouring of the fibres, however, the use of synthetic inorganic pigments has proven to be advantageous. Synthetic inorganic pigments are produced for example by chemical and/or physical conversion (digestion, precipitation, annealing). They include, in particular,
white pigments (titanium dioxide (TiO₂), Pigment White PW 6; zinc sulfide (ZnS), PW 7; zinc oxide (ZnO), PW 4; antimony white (Sb₂O₃), PW 11; lithopone (ZnS/BaSO₄), PW 5; white lead (2PbCO₃×Pb(OH)₂), PW 1),
subordinate white fillers (calcium carbonate, PW 18; talc, PW 26 and barium sulfate, PW 21);
black pigments (manganese black, spinel black and carbon black pigments (graphite carbon);
lustre pigments (absorption pigments, metal pigments or metal effect pigments and pearlescent pigments) and
inorganic coloured pigments (iron oxide pigments, iron blue pigments, ultramarine pigments and, although less suitable on account of their toxicological properties, lead chromate pigments, chromium oxide pigments, cadmium pigments and bismuth vanadate pigments).

Preferred synthetic inorganic pigments are metal pigments or metal effect pigments formed from powdered metals or metal alloys, such as aluminium bronzes (metal: Al) gold bronzes (metal: Cu, Cu—Al or Cu—Zn alloy), silver bronzes (metal: Cu—Zn—Ni), flame-coloured bronzes (metal: oxidised Cu—Zn) and patent bronzes (metal: Cu—Zn—(Ni)+dye).

Further preferred synthetic inorganic pigments are pearlescent pigments which include a number of layers with different refractive index. Examples of such pearlescent pigments are magnesium stearate, zinc stearate and lithium stearate or ethylene glycol distearate or polyethylene terephthalate and pearlescent pigments which consist substantially of mica, titanium dioxide (titanium dioxide mica), bismuth chloride oxide or guanine, and additionally can be coated with coloured oxide layers (for example iron oxides or chromium oxides). Mica-based and mica/metal oxide-based pearlescent pigments are particularly preferred pearlescent pigments as contemplated herein. Micas are sheet silicates. The most important representatives of these silicates are muscovite, phlogopite, paragonite, biotite, lepidolite and margarite. In order to produce the pearlescent pigments in conjunction with metal oxides, the mica, predominantly muscovite or phlogopite, is coated with a metal oxide. Suitable metal oxides are, amongst others, TiO₂, Cr₂O₃ and Fe₂O₃. Interference pigments and coloured lustre pigments are obtained, by appropriate coating, as pearlescent pigments preferred as contemplated herein. These pearlescent pigment types additionally have colour effects besides a sparkly optical effect. Furthermore, the pearlescent pigments usable as contemplated herein can additionally contain a colour pigment which does not derive from a metal oxide.

Very particularly preferred pearlescent pigments are pigments that are marketed by the company Merck under the trade names Colorona®, wherein the pigments Colorona® red-brown (47-57% by weight Muscovite Mica (KH₂(AlSiO₄)₃), 43-50% by weight Fe₂O₃ (INCI: Iron Oxides CI 77491), <3% by weight TiO₂ (INCI: Titanium Dioxide CI 77891), Colorona® Blackstar Blue (39-47% by weight Muscovite Mica (KH₂(AlSiO₄)₃), 53-61% by weight Fe₃O₄ (INCI: Iron Oxides CI 77499)), Colorona® Siena Fine (35-45% by weight Muscovite Mica (KH₂(AlSiO₄)₃), 55-65% by weight Fe₂O₃ (INCI: Iron Oxides CI 77491)), Colorona® Aborigine Amber (50-62% by weight Muscovite Mica (KH₂(AlSiO₄)₃), 36-44% by weight Fe3O4 (INCI: Iron Oxides CI 77499), 2-6% by weight TiO₂ (INCI: Titanium Dioxide CI 77891)), Colorona® Patagonian Purple (42-54% by weight Muscovite Mica (KH₂(AlSiO₄)₃), 26-32% by weight Fe₂O₃ (INCI: Iron Oxides CI 77491), 18-22% by weight TiO₂ (INCI: Titanium Dioxide CI 77891), 2-4% by weight Prussian blue (INCI: Ferric Ferrocyanide CI 77510)), Colorona® Chameleon (40-50% by weight Muscovite Mica (KH₂(AlSiO₄)₃), 50-60% by weight Fe₂O₃ (INCI: Iron Oxides CI 77491)) and Silk® Mica (>98% by weight Muscovite Mica (KH₂(AlSiO₄)₃)) are particularly preferred.

A group of particularly preferred pigments is formed by the colour-giving synthetic iron oxides. Particularly preferred representatives of this substance class are Pigment Brown 6 (CI No 77491), Pigment Red 101 (CI No 77491), Pigment Yellow 42 (CI No 77492), Pigment Black 11 (CI No 77499) and mixtures of these pigments.

Pigments that can be used very particularly preferably are exemplified by a good spray ability in the compositions as contemplated herein, a homogeneous spray pattern, and an extremely low tendency to block the spray nozzles (“clogging”). Cosmetic compositions that are extremely preferred as contemplated herein contain at least one pigment from the group CI12490, CI14700, CI14720, CI15510, CI15985, CI45380, CI47005, CI60730, CI61565, CI73360, CI74160, CI77007, CI77019, CI77288, CI77289, CI77491, and CI77492, wherein the total amount of pigment(s) from this group is from about 0.0001 to about 10% by weight, preferably from about 0.01 to about 9.5% by weight, more preferably from about 0.05 to about 9% by weight, even more preferably from about 0.1 to about 8.5% by weight, and in particular from about 0.25 to about 8% by weight.

The cosmetic compositions particularly preferably contain at least one starch compound. The wording “as further constituent” therefore means “additionally to” all other previously mentioned mandatorily necessary constituents.

Preferred cosmetic compositions contain, as further constituent, from about 1 to about 10% by weight, preferably from about 2 to about 6% by weight, and in particular from about 3 to about 5% by weight of at least one starch compound.

Starch is a reserve carbohydrate which is stored by plants in the form of starch grains (granules) usually from about 1 to about 200 μm in size in various plant parts, for example in tubers or roots, cereal seeds, fruits, and in the pith. Starch belongs to the homoglycan family and is a polycondensation product of D-glucose. Starches that are preferred as contemplated herein are selected from at least one polycondensation product of D-glucose obtained from natural and/or physically modified starch of potatoes, corn, rice, peas, acorns, chestnuts, barley, wheat, bananas, sago, millet, sorghum, oat, rye, beans, batate, maranta or manioc and mixtures thereof. With regard to cleaning power, the use of native and/or physically modified rice starch has proven to be particularly advantageous. Natural starch is understood to mean a starch that is isolated from starch-containing plants and which, after the isolation and purification, was neither physically nor chemically modified. By contrast, physically modified starch is understood to mean a starch that was subjected to at least one physical modification after the isolation. Here, “physical modification” is understood to mean modification with application of pressure and/or heat and/or light. A modification by employing chemical and enzyme reactions, for example the hydrolysis of the starch, however, does not fall under the term “physical modification”. A preferably used physical modification is the application of heat, in particular the boiling of the natural starch. Preferred embodiments of the present disclosure are therefore characterised in that the compositions contain at least one starch selected from chemically and/or physically modified rice starches, in particular from physically modified rice starch. As a result of the use of natural and/or physically modified rice starches in combination with dyes and/or pigments, a particularly high adhesion of the dyes and/or pigments to the starch is attained, and therefore, besides the excellent cleaning power, a long-lasting temporary hair colouring is also made possible.

It is hereby possible and preferred to use pre-coloured and/or pre-pigmented starches, which can be sprayed excellently with the cosmetic compositions. Particularly preferred cosmetic compositions contain ingredients (e) and/or (f) in the form of a starch-dye compound and/or starch-pigment compound, preferably in the form of dyed and/or pigmented rice starch.

It has additionally been found that the composition of the starch particles itself has proven to be relevant for the cosmetic effect. Preferably used particles therefore contain a certain proportion of the native and/or physically modified starch, in particular rice starch. It is thus preferred if the starch-dye compound contains at least one starch, in particular a physically modified rice starch, in a total amount of from about 70 to about 96% by weight, in particular from about 80 to about 94% by weight, in each case in relation to the total weight of the particle (compound). The use of particles which contain a high weight share of physically modified rice starch leads to particularly homogeneous spray patterns and preferred adhesion of the colour to the keratin fibres.

In addition to such compounds formed from starch and colour-giving substances or instead of them, the fibres of the cosmetic compositions can of course also be pre-coloured and/or pre-pigmented. Here, particularly preferred cosmetic compositions are those which contain the ingredients (a) and (e) and/or (a) and (f) in the form of a fibre-dye compound or a fibre-pigment compound, preferably in the form of dyed and/or pigmented fibres.

Preferred cosmetic compositions contain at least one ester oil as further constituent. The group of ester oils includes in particular esters of C₆-C₃₀ fatty acids with C₂-C₃₀ fatty alcohols, preferably monoesters of fatty acids with alcohols with 2 to 24 C atoms. Exemplary ester oils are isopropyl myristate (Rilanit® IPM), isononanoic acid-C16-18-alkyl ester (Cetiol® SN), 2-ethylhexyl palmitate (Cegesoft® 24), stearic acid-2-ethylhexyl ester (Cetiol® 868), cetyloleate, glycerine tricaprylate, coconut fatty alcohol caprinate/caprylate (Cetiol® LC), n-butylstearate, oleylerucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN) and oleic acid decyl ester (Cetiol® V). The use of isopropyl myristate is particularly preferred.

The weight proportion of the ester oil in the total weight of the cosmetic composition is preferably from about 0.01 to about 1% by weight, preferably from about 0.02 to about 0.6% by weight, and in particular from about 0.03 to about 0.2% by weight.

The fibres described at the outset as first essential constituent in the cosmetic compositions can be modified differently. Such modifications relate for example to the length of the fibres described further above or the length-to-diameter ratio thereof.

A further possibility for influencing the technical and cosmetic properties of the compositions lies in the modification of the surface properties of the used fibres.

Hydrophobised fibres can preferably be used. The hydrophobisation (impregnation) provides the fibres with a water-repelling property. For example, paraffins or waxes, but also film-forming silicones are suitable as hydrophobising agents.

Depending on the intended purpose of the cosmetic composition, it can be advantageous if the fibres have a certain oil absorption capacity. This oil absorption capacity can be influenced by employing the previously described hydrophobisation of the fibres. The use of hydrophobised or non-hydrophobised fibres with an oil absorption capacity above about 0.2 g jojoba oil per gram of fibres is preferred. The use of fibres with an oil absorption capacity (20° C.) of from about 0.2 to about 0.8 g jojoba oil/g, preferably of from about 0.3 to about 0.7 g jojoba oil/g, particularly preferably of from about 0.4 to about 0.6 g jojoba oil/g, is particularly preferred.

For example, in order to enable a uniform suspension of the fibres in the cosmetic composition or in order to increase the cosmetic effect of the composition, the compositions can comprise surfactant as a further optional constituent. The use of non-ionic or cationic surfactants is preferred.

Preferred cationic surfactants are selected from the group of quaternary ammonium compounds. Preferred quaternary ammonium compounds are, in turn, ammonium halides, in particular chlorides and bromides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammoniumchloride and tricetyl methyl ammonium chloride, and the imidazolium compounds known under the INCI names Quaternium-27 and Quaternium-83. The use of cetyl trimethyl ammonium chloride is very particularly preferred.

The weight proportion of the surfactant in the total weight of the cosmetic composition is preferably from about 0.05 to about 2% by weight, preferably from about 0.1 to about 1.5% by weight, and in particular from about 0.2 to about 1% by weight.

In an alternative embodiment hydrophilised fibres can be used in the cosmetic compositions. Corresponding fibres have an increased capacity for water absorption in comparison to the non-hydrophilised fibres.

The water absorption capacity (about 20° C.) of preferred fibres, particularly of preferred hydrophilised fibres, lies preferably above about 5.0% by weight, preferably above about 7.0% by weight, and in particular above about 9.0% of the net weight of the fibres.

The hydrophilisation of the fibres improves their water absorption capacity, amongst other things. Corresponding fibres are therefore used in particular in freshening cosmetic compositions.

In a further alternative embodiment, preferred cosmetic compositions contain at least one fragrance.

In particular, additional nourishing substances are optional further suitable active substances or auxiliaries that can be contained in the cosmetic compositions.

As nourishing substance, the product can contain at least one protein hydrolysate and/or a derivative thereof, for example. Protein hydrolysates are product mixtures which are obtained by acid-catalysed, base-catalysed or enzymatically catalysed breakdown of proteins. The term “protein hydrolysates” is understood as contemplated herein to also mean total hydrolysates and also individual amino acids and derivatives thereof as well as mixtures of different amino acids. The molecular weight of the protein hydrolysates that can be used as contemplated herein lies between about 75, the molecular weight for glycine, and about 200,000, and the molecular weight is preferably from about 75 to about 50,000, and very particularly preferably from about 75 to about 20,000 Dalton.

As nourishing substance, the cosmetic composition can also contain at least one vitamin, a provitamin, a vitamin precursor and/or one of the derivatives thereof. Here, vitamins, provitamins and vitamin precursors that are usually assigned to the groups A, B, C, E, F and H are preferred.

Further nourishing substances are panthenol, caffeine, nicotinamide and sorbitol.

The cosmetic compositions may also contain at least one plant extract, but also mono- or oligosaccharides and/or lipids as nourishing substance.

In particular, aerosol-dispensing containers are suitable for the dispensing and application of the cosmetic compositions. A further subject matter of the application relates to the corresponding cosmetic product, comprising
a) a cosmetic composition as contemplated herein
b) an aerosol-dispensing container.

The term “aerosol-dispensing container” is understood to mean pressurised containers inside which a higher gas pressure prevails than outside the container and from which a gas flow can be discharged via a valve. The aerosol-dispensing container, in other words, is a pressurised container with the aid of which a product (for example a cosmetic composition) can be dispensed via a valve by way of the internal gas pressure of the container.

The cosmetic compositions can be produced in the conventional manner. Generally, all constituents of the cosmetic composition with the exception of the propellant are filled into a suitable pressure-tight container. This is then sealed using a valve. Lastly, the desired amount of propellant is filled by way of conventional techniques.

Vessels made of metal (aluminium, tinplate, tin), protected or non-splintering plastic or glass, which is coated externally with plastic, are potential pressure-tight containers, wherein the pressure tightness and breaking strength, corrosion resistance, ease of filling and also aesthetic considerations, ease of handling, printability, etc. all play a role in the selection of such vessels. Special interior protective coatings ensure the corrosion resistance in relation to the composition packaged inside the pressurised container. The used valves particularly preferably have an internally-coated valve disc, wherein coating and valve material are compatible with one another. If aluminium valves are used, their valve discs can thus be coated on the inside, for example with a Micoflex coating. If tinplate valves are used as contemplated herein, their valve discs can thus be coated on the inside, for example with PET (polyethylene terephthalate).

A multi-chamber dispenser can also be used as aerosol-dispensing container. The multi-chamber dispenser can also be designed such that one chamber is filled with the compressed propellant and another chamber is filled with the rest of the constituents of the composition as contemplated herein. A multi-chamber dispenser of this kind is for example what is known as a bag-in-can packaging.

The spray rate of the cosmetic product is preferably from about 6.5 to about 10.0 g/10 s.

A further subject of this application is a method for temporarily reshaping keratin fibres, in particular human hair, in which method a cosmetic composition as contemplated herein is applied to keratin fibres. That stated above also applies, mutatis mutandis, for the constituents of this composition, weight proportions thereof and preferred embodiments thereof.

A last subject of this application is a method for increasing the thickness of keratin fibres, in particular human hair, in which a cosmetic composition as contemplated herein, containing a copolymer with the INCI name Castor Oil/IPDI Copolymer, is applied to keratin fibres. That stated above also applies, mutatis mutandis, for the constituents of this composition, weight proportions thereof and preferred embodiments thereof.

The following example is intended to explain the subject matter of the present disclosure, without limiting it in any way.

### EXAMPLE

TABLE 1
Composition of five compositions as contemplated herein (values
in % by weight active substance, unless stated otherwise)

| Ingredient | 1 | 2 | 3 | 4 | 5 |
|---|---|---|---|---|---|
| Hydroxypropyl | 0.05 | 0.12 | 0.05 | 0.12 | 0.12 |
| methylcellulose | | | | | |
| Cellulose fibres | 0.4 | 0.2 | 0.4 | 0.2 | 0.2 |
| (Bambusa | | | | | |
| Arudinacea | | | | | |
| Stem Powder) | | | | | |
| Eurocryl BC 4365* | 3 | 1.5 | 1.5 | 0.75 | 0.75 |
| Castor Oil/IPDI | 0.8 | 1.2 | — | — | — |
| Copolymer | | | | | |
| Polyvinyl Acetate | — | 0.55 | — | 0.55 | 0.55 |
| PEG-32 | — | 0.5 | — | — | 0.5 |
| Silica Dimethyl Silylate | — | 0.5 | — | 0.3 | — |
| K-Flex DU-320 100** | — | — | 1.2 | 1.23 | 1.23 |
| Crotonic Acid/Vinyl | — | — | — | 0.5 | — |
| C8-12 Isoalkyl | | | | | |
| Esters/VA/Bis- | | | | | |
| Vinyldimethicone | | | | | |
| Crosspolymer | | | | | |
| Perfume | 0.1 | 0.06 | 0.1 | 0.06 | 0.06 |
| Dimethyl ether | 70 | 70 | 70 | 70 | 70 |
| Ethanol | to 100 | to 100 | to 100 | to 100 | to 100 |

*INCI: Aqua, Ethylhexyl Acrylate/Methyl Methacrylate (active content: 50% by weight)
**Oligomer of a urethane diol (ex King)

The compositions as contemplated herein were applied to test subjects in order to fix the shape of the hairstyle. The test subjects reported a positive hair feel, i.e. not too rough or sticky. The hair achieves a strong hairstyle hold. The hair of the test subjects looked fuller. In particular when applying compositions 1 and 2, the hair of the test subjects also felt fuller. Compositions 2, 4 and 5 additionally had a texturising effect.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for the cosmetic treatment of keratin fibres, said composition comprising:
(a) fibres;
(b) ethanol;
(c) at least one polymer, comprising an acrylate copolymer; and
(d) propellant.

2. The cosmetic composition according to claim 1, wherein the acrylate copolymer is selected from the group of compounds with the INCI name Ethylhexyl Acrylate/Methyl Methacrylate Copolymer

3. The cosmetic composition according to claim 1, wherein the at least one polymer comprises further polymers selected from the group consisting of polyvinyl acetate, a copolymer with the INCI name Castor Oil/IPDI Copolymer, polyurethanes, cellulose ethers, polyethylene glycols, a copolymer with the INCI name Acrylates/Hydroxyesters Acrylates Copolymer, copolymers of methylvinyl ether and maleic acid anhydride, and a copolymer with the INCI name Crotonic Acid/Vinyl C8-12 Isoalkyl Esters/VA/Bis-Vinyldimethicone Crosspolymer.

4. The cosmetic composition according to claim 1, wherein the composition comprises a cellulose ether as further polymer.

5. The cosmetic composition according to claim 1, wherein the cosmetic composition comprises a copolymer with the INCI name Castor Oil/IPDI Copolymer as further polymer.

6. The cosmetic composition according to claim 1, wherein the cosmetic composition further comprises a hydrophobised metal oxide powder.

7. The cosmetic composition according to claim 1, wherein the cosmetic composition further comprises an oligomer of a urethane diol.

8. The cosmetic composition according to claim 1, wherein the weight proportion of the at least one polymer in the total weight of the cosmetic composition is from about 0.1 to about 10% by weight.

9. The cosmetic composition according to claim 1, wherein the fibres are selected from the group consisting of cellulose fibres, cotton fibres, flax fibres, bamboo fibres and kapok fibres.

10. The cosmetic composition according to claim 1, wherein the fibres are selected from the group consisting of polyamide fibres, polyester fibres, polylactic acid fibres and polyhydroxyalkanoate fibres.

11. The cosmetic composition according to claim 1, wherein the fibres have a length of from about 1 μm to about 200 μm.

12. The cosmetic composition according to claim 1, wherein the fibres have a length of from about 5 μm to about 100 μm.

13. The cosmetic composition according to claim 1, wherein the weight proportion of the ethanol in the total weight of the cosmetic composition is from about 10 to about 40% by weight.

14. A cosmetic product, comprising
a) a cosmetic composition according to claim 1 contained in
b) an aerosol-dispensing container.

15. A method for increasing the thickness of keratin fibres, comprising applying the cosmetic composition according to claim 1 to keratin fibres.
